(19) United States
(12) Patent Application Publication
(10) Pub. No.: US 2022/0219296 A1
(43) Pub. Date: Jul. 14, 2022

(54) TRACKABLE MARKER DISPENSER

(71) Applicant: DEPUY IRELAND UNLIMITED COMPANY, RINGASKIDDY (IE)

(72) Inventors: Andrew BAILEY, LEEDS (GB); Christopher HUNT, LEEDS (GB); Louise WRIGHT, LEEDS (GB)

(21) Appl. No.: 17/610,341

(22) PCT Filed: Apr. 22, 2020
(86) PCT No.: PCT/EP2020/061224

§ 371 Date: Nov. 10, 2021

(30) Foreign Application Priority Data
May 16, 2019 (GB) 1906904.6

(51) Int. Cl. B25B 23/06; A61B 50/20; A61B 50/30

(52) U.S. Cl. CPC B25B 23/065; A61B 50/20; A61B 2090/031; A61B 2050/3013; A61B 50/30

(57) ABSTRACT

A dispenser for dispensing and attaching individual markers to a reference array of a tracking system is described. A housing defines a cavity extending along a longitudinal axis. The housing has a dispensing end defining a dispensing aperture. A plurality of markers are stacked within the cavity. Each marker has a marker attachment feature by which the marker is attachable to an attachment site of a reference array. The dispensing end of the housing provides a retainer which exerts a retaining force on a bottom most marker sufficient to retain the plurality of markers within the cavity but permitting the bottom most marker to be released from the cavity when the bottom most marker has been attached by its attachment feature to the attachment site of the reference array.

The present invention relates to a dispenser and a method of use of the dispenser, and in particular to a dispenser for markers detectable by a tracking system used by a computer assisted surgery system.

Computer assisted surgery (CAS) systems are generally known and often use a tracking system which determines the location and/or orientation of various items in the operating theatre, including the body parts or the patient, surgical equipment, tools, instruments and implants and such like. Some CAS systems may also include robotic systems.

The items being tracked have markers which are detectable by the tracking system attached to them. The exact nature of the markers may vary depending on the tracking system. Some tracking systems may use wired markers and other may use wireless markers. Wireless markers are often active markers, which themselves emit some form of signal which is detected by the tracking system, or passive markers which reflect some form of signal emitted by the tracking system. For example, acoustic signals, magnetic signals and light signals, particularly infra-red light, may be used by wireless tracking systems.

At some stage, the markers are attached to the items that will be tracked. Sometimes, this involves attaching a reference array to the item being tracked and wherein the reference array includes a number of markers with a particular configuration, so that that particular reference array, and hence the item it is attached to, can be distinguished from other reference arrays that may be detectable by the tracking system.

Through repeated use, the markers may become damaged or otherwise require replacement.

It may be common for markers to be single use to ensure that they give the best performance for a surgery, and also because it is hard to reprocess them without effecting their performance. This means that it is common to attach new markers to all the arrays at the start of surgery.

When markers are disposable, then it may be necessary to replace the markers of a reference array.

A reference array may need to be modified or reconfigured by changing the relative locations of the markers on the reference array.

If a reference array becomes damaged, for example bent out of shape, then a replacement reference array may be needed and markers may need attaching.

If a further reference array is needed, for example because an uncommon instrument is to be used and needs to be tracked, then a new reference array may need assembling.

Sometimes, a marker may get dirty during surgery, and so will need to be replaced mid surgery.

Hence, there are a number of instances in which it would be beneficial to be able more efficiently to attach markers to a reference array.

A first aspect of the invention provides a dispenser for dispensing and attaching individual markers to a reference array of a tracking system, the dispenser comprising: a housing defining a cavity therein extending along a longitudinal axis of the housing and the housing having a dispensing end defining a dispensing aperture of the cavity; and a plurality of markers stacked within the cavity, each marker having a marker attachment feature by which the marker is attachable to an attachment site of a reference array, and wherein the dispensing end provides a retainer exerting a retaining force on a bottom most marker sufficient to retain markers within the cavity but permitting the bottom most marker to be released from the cavity via the dispensing aperture when the bottom most marker has been attached by its attachment feature to the attachment site of the reference array.

The retainer may include at least one sprung member. The retainer may include a plurality of sprung members. Adjacent pairs of sprung members may each define a respective slot between them. Free ends of the sprung members may define the dispensing aperture.

The dispensing end may be deformable to provide the retainer. The dispensing end may deform by flexing.

The marker attachment feature may be a thread. The thread may be a male thread or a female thread.

The attachment site may include a thread. The thread may be a male thread or a female thread.

Each marker may include an anti-rotation feature which interacts with a feature of an inner wall defining the cavity to prevent rotation of the marker relative to the housing.

Each marker may include a plurality of anti-rotation features which interact with a plurality of features of an inner wall defining the cavity to prevent rotation of the marker relative to the housing

The or each anti-rotation feature may interact with the or each feature via friction or friction alone.

The anti-rotation feature may be one of a male formation and a female formation and the feature of the inner wall may be the other of the male formation and the female formation.

The or each anti-rotation feature may comprise one or a plurality of male formations. The feature of the inner wall may comprise one or more female formations extending along the longitudinal axis of the housing.

The male formation may be a rib or a ridge. The female formation may be a groove or a slot. The female formation may have an upper part in the form of a groove and a lower part in the form of a slot.

The or each feature of the inner wall may extend along the longitudinal axis all the way to the dispensing aperture.

The dispenser includes a torque limiting mechanism. The torque limiting mechanism may interact with only the bottom most marker. The retainer may provide the torque limiting mechanism.

The torque limiting mechanism may permit the dispenser to rotate relative to a bottom most marker when a torque or rotational force being applied to the bottom most marker by the housing exceeds a limit.

The housing may comprise an upper part and a lower part. The upper part may define the cavity. The lower part may be movable along the longitudinal axis toward the upper part from a first position to a second position. The lower part may extend beyond the dispensing aperture in the first position and the lower part may not extend beyond the dispensing aperture in the second position.

The marker attachment feature may be an adhesive material. The marker attachment feature may be a push fit connection or a snap fit connection.

A spacer may be provided between each adjacent pair of markers. The or each spacer may be in the form of a ring or annulus.

Each marker may comprise a holder and a detection part which is detectable by the tracking system. The holder may provide the marker attachment feature

Each marker may include a circular, annular, spherical or hemi-spherical or spherical cap shaped reflector, transmitter or emitter of radiation. Said radiation may be light. The radiation may be infrared light.

The dispenser may further comprise a resilient element arranged to bias the plurality of markers toward the dispensing aperture. The resilient element may be arranged between an upper part of the cavity and a top most marker of the markers in the cavity. The resilient element may be a spring.

An upper end of the housing may include a drive connection by which a drive tool can be attached to the dispenser. The drive tool may be a power tool.

The dispenser may hold at least three markers. The dispenser may hold up to twenty markers. The number of markers may be in the range of three to twenty.

The number of markers in the dispenser may be an integer multiple of three or four. The number of markers may be three, six, nine, twelve, fifteen or eighteen. The number of markers may be four, eight, twelve, sixteen or twenty.

The dispenser may further include a packet within which the dispenser is contained. The packet may be made from a plastic or polymer. For example, nylon may be used or Polyethylene terephthalate (PET).

The dispenser and/or the markers may be sterile.

A second aspect of the invention provides a package comprising: a plurality of dispensers according to the first aspect of the invention and a container holding the plurality of dispensers. The container may be a box or carton or similar.

A third aspect of the invention provides a kit of parts comprising: one or more dispensers according to the first aspect or the package of the second aspect; and one or more reference arrays, each reference array having a plurality of attachment sites and wherein each of the plurality of attachment sites has an array attachment feature to which the respective marker attachment features are respectively attachable.

A fourth aspect of the invention provides a method of attaching markers to a reference array for a tracking system using a dispenser holding a plurality of markers, the method comprising: presenting a dispensing end of a dispenser holding a plurality of markers to an attachment site of the reference array; attaching a bottom most marker within the dispenser to the attachment site using the dispenser; and withdrawing the dispenser to release the distal most marker from the dispenser while retaining the marker attached to the attachment site.

The method may further comprise repeating the method for each of a plurality of attachment sites of the reference array.

The method may further comprise repeating the method for each of a plurality of reference arrays.

The method may further comprise rotating the dispenser to attach the distal most marker to the attachment site.

The method may further comprise attaching a power tool to the dispenser and/or using a power tool to rotate the dispenser.

The method may further comprise removing the or each dispenser from a sterile packet.

The method may further comprise taking the or each dispenser from a container holding a plurality of dispensers.

Embodiments of the invention will now be described in detail, by way of example only, and with reference to the accompanying drawings, in which:

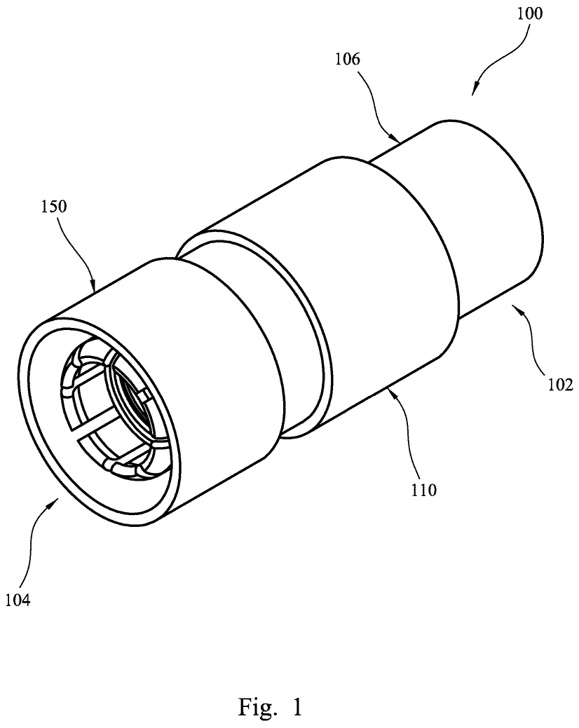

FIG. 1 shows a perspective view of a dispenser according to a first embodiment of the invention;

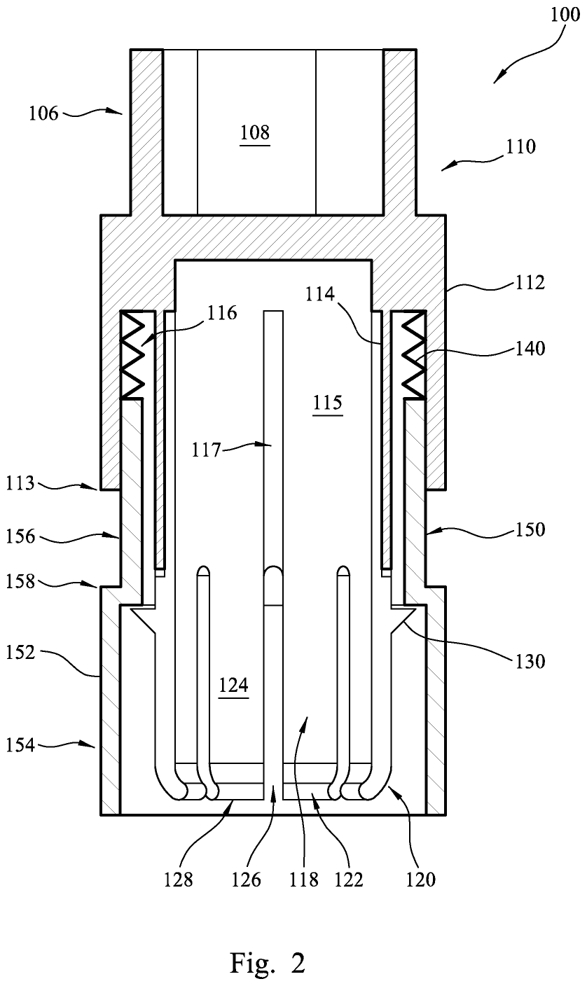

FIG. 2 shows a cross sectional view of the dispenser shown in FIG. 1;

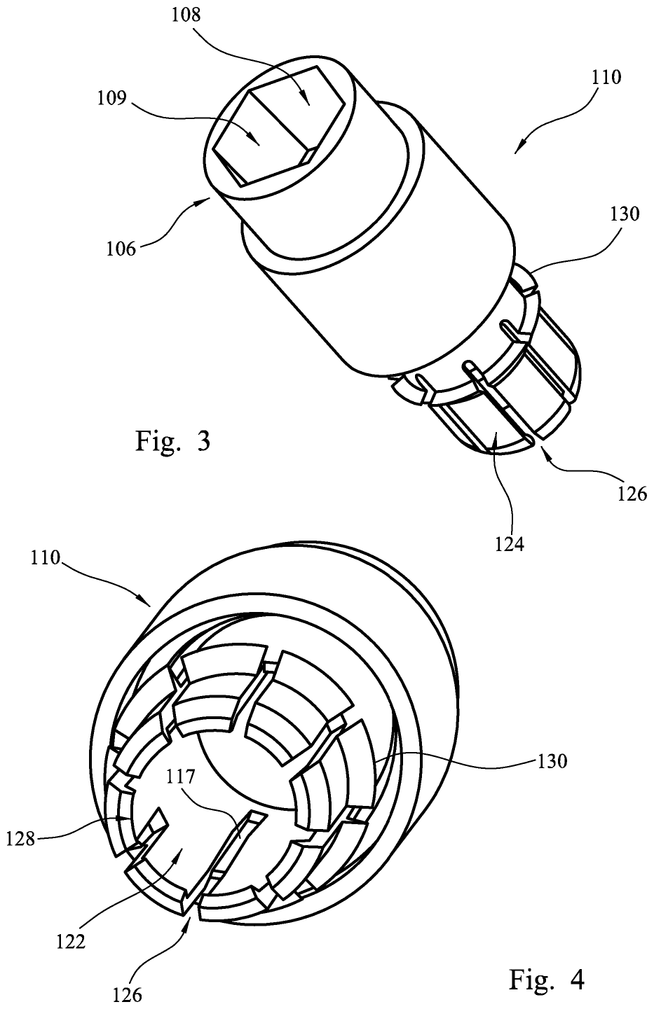

FIG. 3 shows a perspective view of an upper part of the dispenser from above;

FIG. 4 shows a perspective view of the upper part of the dispenser from below;

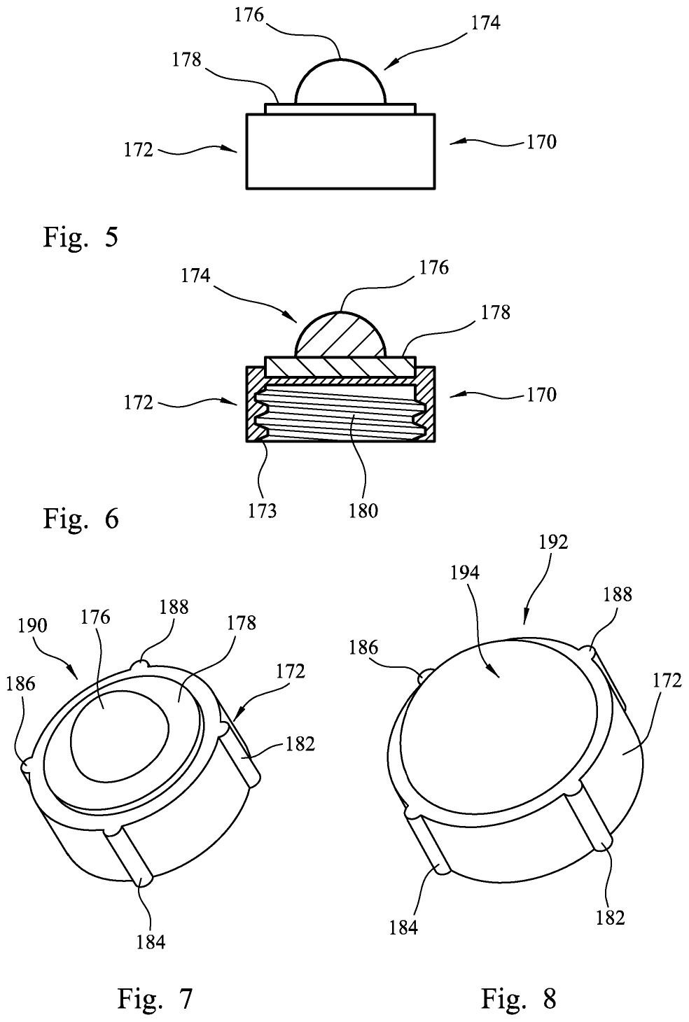

FIG. 5 shows a side view of a first embodiment of a marker which can be used with the dispenser;

FIG. 6 shows a cross sectional view of the marker shown in FIG. 5;

FIG. 7 shows a perspective view of a second embodiment of a marker which can be used with the dispenser;

FIG. 8 shows a perspective view of a third embodiment of a marker which can be used with the dispenser;

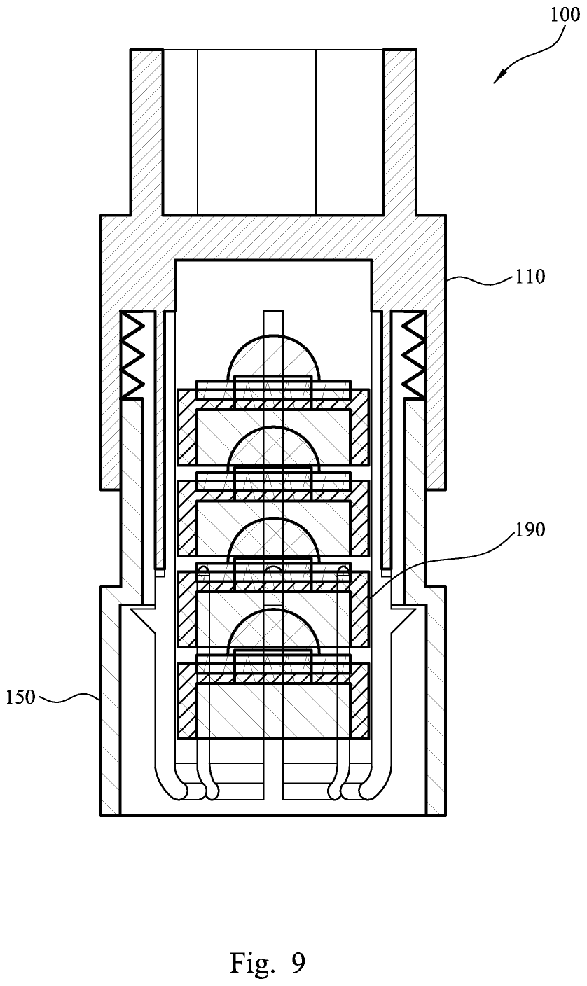

FIG. 9 shows a cross sectional view of the dispenser with a plurality of markers stacked therein;

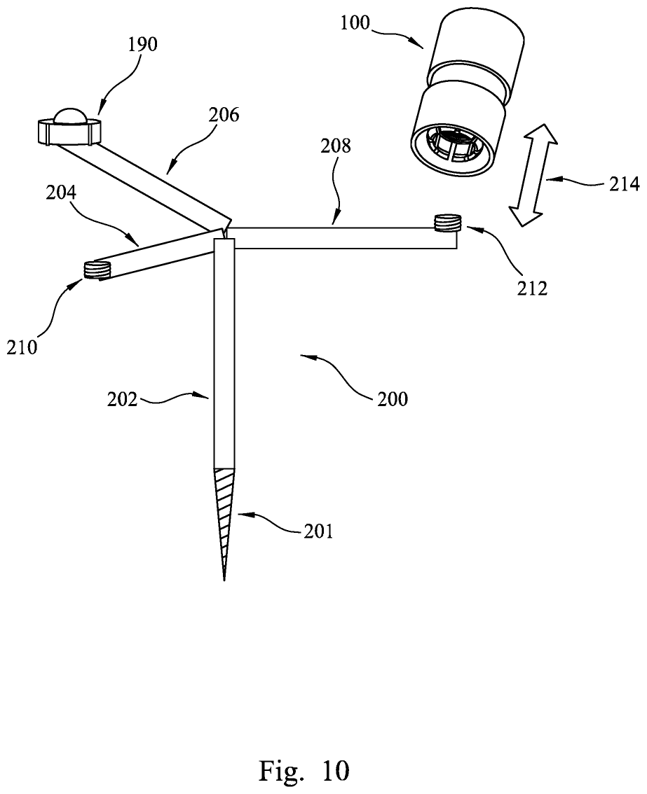

FIG. 10 shows a schematic diagram illustrating use of the dispenser to attach markers to a reference array;

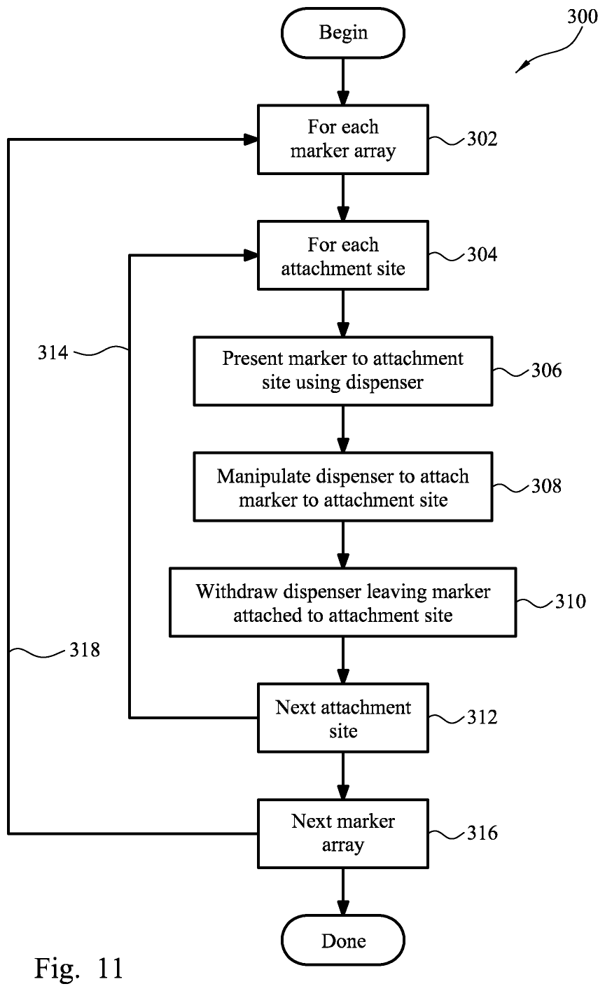

FIG. 11 shows a flow chart illustrating a method of use of the dispenser to attach markers to one or more reference arrays according to an embodiment of the invention; and

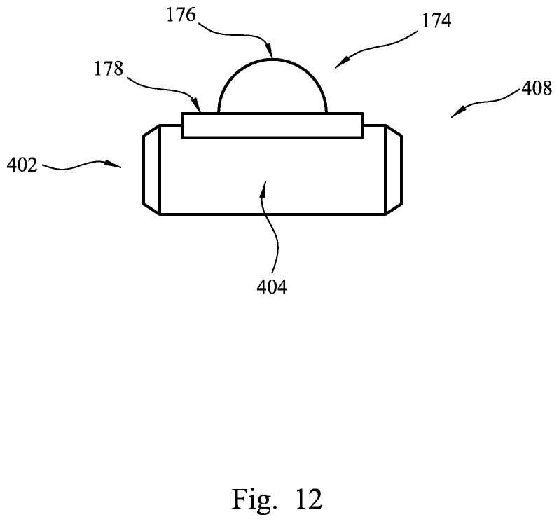

FIG. 12 shows a fourth embodiment of a marker which can be used with the dispenser of the invention.

In the Figures, similar items share common reference signs unless indicated otherwise. With reference to FIG. 1, there is shown a perspective view of a dispenser 100 according to an embodiment of the invention. The dispenser 100 has a generally tubular construction and has an upper, connecting end 102 and a lower, dispensing end 104. The dispenser 100 generally has a two part construction and includes an upper part 110 and a lower part 150. The connecting end 102 includes a connection formation 106 by which a drive tool, for example a manual tool or a power tool, may be releasably attached to the dispenser to rotate the dispenser in use, as described in greater detail below. However, in other embodiments, no drive connection need be provided. In the illustrated embodiment the connection formation 106 generally has the form of a cylindrical wall having an inner surface comprising a plurality of flat surfaces, e.g., surface 108, which defines a generally hexagonal cavity for receiving a hex head of a drive tool. However, other forms of connection formation may also be used so long as they allow a rotational force to be applied to the dispenser 100 in use.

FIG. 2 shows a cross sectional view of the dispenser 100. The dispenser 100 generally comprises a housing within which a plurality of markers are stored, as further illustrated in FIG. 9, and from which markers can individually be dispensed. As best illustrated in FIG. 2, the housing including a first or upper part 110 and a second or lower part 150. The upper part 110 of the housing has a generally cylindrical form and includes an outer wall 112 and an inner wall 114 with an annular cavity 116 defined there between. An inner surface 115 of the inner curved wall 114 defines a cylindrical cavity 118 extending generally along a longitudinal axis of the dispenser 100. Four grooves, e.g., groove 117, extending along the longitudinal axis of the dispenser 100 are defined in the inner surface 115. The four grooves are generally equi-angularly disposed about the longitudinal axis, for example at approximately 0°, 90°, 1800 and 270° respectively.

A lower end 120 of the inner wall 114 defines a lower dispensing aperture 122 or mouth of the cavity 118. The lower end of the inner wall 20 also provides a retainer for retaining markers within the cavity 118. In the illustrated embodiment, the retainer is in the form of a plurality of limbs, e.g. limb 124, defining slots, e.g. 126, between adjacent pairs of limbs. A free end of each limb, e.g. 128, is directed inwardly toward the centre of the dispenser such that the diameter of the dispensing aperture 122 is slightly less than the diameter of the markers when located within the cavity 118.

As illustrated in FIG. 2, the four grooves, e.g. groove 117, within the inner wall 115 are each aligned with a corresponding slot, e.g. slot 126, to allow markers to be dispensed from the dispenser and to provide a mechanism by which to transfer torque from the dispenser to the markers within it as described in greater detail below. It will be noted that the grooves, e.g. 117, are closed whereas the slots, e.g. 126, are open.

The slots 126 between the limbs 124 allow the limbs 124 to flex outwardly thereby increasing the diameter of the distal aperture 122 to permit markers to be released from the dispenser. The limbs 124 are resilient and will return to the configuration illustrated in FIG. 2 after releasing a marker 170 thereby retaining a subsequent marker within the dispenser in use. Further, the ability of the limbs 124 to flex may provide a torque limiting function in some embodiments as this permits corresponding ribs on the markers to escape from the slots 126 to permit the dispenser to rotate relative to the marker when a marker has been sufficiently secured in use as described below.

A shoulder 130 is provided on an outer surface of the second wall 114 and extending around the circumference thereof.

The second or lower part 150 of the housing has a generally tubular construction and includes a cylindrical wall 152 having a greater diameter at a lower end 154 and a lesser diameter at an upper end 156. A shoulder 158 connects the upper 156 and lower 154 parts of the second part 150 of the housing. The upper wall 156 is received within the annular aperture 116 of the upper part 110 of the housing. A spring or other biasing element 140 is provided within the annular aperture 116 and between the upper and lower parts of the housing and acts to urge the lower part of the housing 150 in a direction away from the upper part of the housing 110 and toward the lower end 104 of the dispenser. The shoulder 158 of the lower part abuts the shoulder 130 of the upper part and provides a stop to limit the travel of the lower part relative to the upper part of the housing, while permitting a bottom most marked to be dispensed.

As illustrated in FIG. 2, the lower part 150, under action of spring 140, adopts a first position in which the lower part 150 extends beyond the dispensing mouth 122. As described in greater detail below, in use, the lower part 150 can travel toward the upper part 110 when sufficient force is applied to overcome the action of spring 140 so as to expose the dispensing mouth 122. The lower part 150 can move toward the upper part 110 by an amount approximately equal to the height of a marker to be dispensed, as controlled by the position of shoulder 158 relative to the lower end 113 of outer wall 112. The movement of the lower part relative to the upper part can assist in avoiding cross threading of a threaded attachment formation in embodiments using threaded attachment formations.

FIG. 3 shows a perspective view generally from above of the upper part 110 of the dispenser 100, illustrating the connection formation 106 and hexagonal cavity 109 defined by the inner flat walls 108 thereof, as well as the plurality of slots, e.g. slot 126, defined between adjacent limbs, e.g. limb 124.

FIG. 4 shows a perspective view generally from below of the upper part 110 of the dispenser 100, illustrating a groove, e.g. 117, aligned with a slot 126, and also the dispensing mouth 122 defined by the free ends 128 of the limbs 124

The upper 110 and lower 150 parts of the dispenser may be made from plastic or metal. For example, plastic may be used for disposable versions of the dispensers. Suitable plastics include various polymers including Polyetheretherketone (PEEK), Polyoxymethylene (POM), glass reinforced Nylon, Polyphenylsulfone (PPSU) and similar. For reusable versions of the dispensers, metal may be used instead, including stainless steel, such as 17-4 stainless steel for example.

FIG. 5 shows a side elevation of a first embodiment of a marker 170 which may be used with the dispenser 100. FIG. 6 shows a transverse cross section view of the marker 170. Although only a single marker is illustrated in FIG. 5, in practice, a plurality of markers arranged in a stack are provided within cavity 118. The plurality of markers are free to travel within the cylindrical cavity 118 and a bottom most marker will be positioned adjacent the dispensing 122 but retained within the dispenser.

As illustrated in FIGS. 5 and 6, each marker 170 includes a holder 172 and a detection part 174 which is detectable by the tracking system. The detection part 174 includes a transparent hemispherical component 176 with a coating of an infra-red reflective material on a back surface and mounted on a disc 178. The detection part functions similarly to a road ‘cats eye’. The holder 172 includes an upper recess within which the disc 178 is received and has a side wall 173 defining an internal thread 180 which provides a marker attachment feature for the marker 170.

As discussed above, a plurality of markers 170 are provided within the cavity 118 in a stacked formation. A benefit of the internal thread 180 is that the detection part 174 of a lower marker can be received within the holder part 172 of an adjacent upper marker when stacked. Hence, the dispenser may hold, for example, six markers within the cavity 118.

The marker 170 does not include ribs on an outer surface of the holder part 172 and so friction between the holder part and the inner surface 115 of the inner wall 114 may be used to transfer rotation from the dispenser to the marker. Also, the magnitude of the frictional force between the dispenser and the marker holder may provide a torque limiter to allow relative rotation between the dispenser and marker when the marker has been sufficiently secured in use.

FIG. 7 shows a perspective view of a second embodiment of a marker 190, generally similar to the first embodiment 170, except that the marker holder 172 includes four equi-angularly spaced ribs, 182, 184, 186 and 188 on an outer surface of the marker holder 172. These four ribs are arranged to co-operate with the correspondingly positioned grooves within the inner wall of the dispenser to provide a mechanism to transfer rotation from the dispenser to the markers within the dispenser. The markers 190 can slide along the longitudinal axis of the dispenser, guided by the ribs sliding within the slots, as markers are dispensed. When a maker 190 has been sufficiently secured, then the flexible limbs 124 can flex outwardly and ride over the ribs 182-188, to provide a torque limitation mechanism, and allow the dispenser to continue to rotate relative to the stationary marker.

FIG. 8 shows a perspective view of a third embodiment of a marker 192, generally similar to the second embodiment 190, but in which the detection part comprises a larger hemispherical component 194 with its convex surface covered with a reflective material which may be painted onto the hemispherical member. Suitable reflective materials are provided by the 3M company under the trade mark SCOTCHLITE Other types of markers with different detection parts may also be used. Other detection parts may simply be in the form of circles or discs of a reflective material or a material with a reflective coating.

FIG. 9 shows a cross sectional view of the dispenser 100 with a stack of four markers 190 located within the inner cavity 118.

Use of the dispenser 100 to dispense and attach markers to a reference array will now be described in greater detail with reference to FIGS. 10 and 11.

FIG. 10 shows a schematic diagram showing the dispenser 100 being used to attach markers 190 to a reference array 200.

The reference array 200 includes a support 202 having a bone anchor 201 or some other attachment formation, for example a clamp or clip, by which the reference array 200 can be attached to a patient's bone or an instrument in use. A first 204, second 206 and third 208 arms extend from the support 202 and each arm includes a threaded boss 210, 212, at a free end thereof. The threaded bosses 210, 212, each provide a separate attachment site on the reference array to which a respective marker can be attached using the dispenser 100. As illustrated in FIG. 10, a first marker 190 has already been attached to arm 206. FIG. 11 shows a flow chart illustrating a method of use 300 of the dispenser 100 as illustrated in FIG. 10. The user may be provided with a container or package holding a plurality of dispensers 100. Each dispenser is pre-loaded with a plurality of markers and each dispenser, and its contents, may itself be sterile and may also be provided in a sterile packet that provides a sterile barrier and which the user removes before using the sterile dispenser 100. The packet, dispenser and markers may be sterilised with gamma radiation, for example. The packet may take various forms. For example, the packet may be provided as a double nylon peel pouch, or in a PET double blister pack with a polyethylene fiber lid. A suitable material for the lid is provide under the trade mark TYVEK by the DuPont company. As the dispenser at least is sterile, there is no need to provide individual packaging for each marker.

The dispenser 100 is provided pre-loaded with a plurality of markers. Reference arrays commonly require at least three markers and therefore the dispenser storing an integer multiple of three markers, e.g. 3, 6, 9, can be preferable. However, a greater or lesser number of markers may be provided depending on the circumstances.

Any drive tool, for example a power tool, maybe connected to the dispenser after it has been removed from its sterile packet. At 302, an initial marker array is selected. Then at 304 an initial attachment site for the current marker array is selected. At 306, a user presents the dispensing end of the dispenser 100 to the attachment site by moving the dispenser 100 toward the attachment site along the direction generally of arrow 214. The plane of the mouth on the lower part 150 of the housing is generally perpendicular to the longitudinal axis of the dispenser. The lower part 150 of the dispenser is rested against the arm 208 of the reference array and with the bottom most marker generally aligned with the centre of the attachment site 212. By pressing on the upper part 110 of the housing, the distal aperture 122 and bottom most marker 190 may be moved toward the attachment site generally along the longitudinal axis of the dispenser and generally parallel to the axis of the threaded boss 212 thereby helping to avoid cross threading.

At 308, the user may rotate the dispenser 100 to engage the threaded boss 212 and internal thread 180 of the bottom most marker 190 so as to attach the marker to the attachment site. In some embodiments, this may be done using a power tool attached to the dispenser 100 or a manual tool. In other embodiments, the user may simply twist the dispenser manually.

Once the bottom most marker has been securely attached to the attachment site, the bottom most marker will become stationary and the dispenser can rotate relative to the bottom most marker either owing to the frictional force being overcome or because of the members 124 flexing a riding over the ribs 182-188 on the marker holder. The dispenser may be withdrawn away from the attachment site, generally along the direction of arrow 214. The flexible elements 124 provide a retaining force sufficient to retain the markers 190 within the cavity 118. However, when the dispenser is withdrawn from the attachment site, the elements 124 may deform outwardly, thereby releasing the bottom most marker 190 from the dispenser 100. As soon as the bottom most marker has been dispensed, the elements 124 spring back into position and prevent a next marker from being released.

At 312, a next attachment site of the reference array 200 may be selected and the method returns, as illustrated by the line 314, to step 304. The method may then repeat so as to attach another marker to a next attachment site of the current reference array. Once markers have been attached to all of the attachment sites, at 316, a next reference array may be selected and the method may repeat for a next reference array, as illustrated by return line 318. However, it is not essential that the method be repeated for multiple reference arrays and there is benefit in using the dispenser of the invention irrespective of the number of reference arrays and number of attachment sites. It will be appreciated that if at any time all of the markers within the dispenser currently being used have been dispensed, then the user can simply take another dispenser from the package of dispensers, remove the sterile packet and then use a next dispenser to attach a next marker.

Use of the dispenser 100 of the invention may provide a number of benefits.

For example, use of the dispenser helps to avoid handling difficulties in manually attaching markers to a reference array. The markers may be reasonably small and difficult to handle manually.

This may be exacerbated if used in theatre if theatre staff are wearing gloves.

Further, it may help to avoid damage to the surface of the marker which may need to have specific optical properties in order to be reliably tracked.

Further, there is no need to remove markers from external packaging that might otherwise be provided to protect the surface of the markers. Furthermore, manual or power tools may be attached to the dispenser rather than having to rely on manual attachment of the marker to the reference array which may also be difficult in theatre when using gloves.

Furthermore, the dispenser 100 may improve the reliability with which markers can be attached to a reference array, for example by avoiding marker arrays being dropped or lost and thereby helping to reduce wastage. This may also improve safety by avoiding marker arrays being dropped into the operating site.

Various modifications and variations of the dispenser 100 and markers 170, 190, 192 will now be described.

In some embodiments, a spring or other biasing device may be provided within the cavity 118 to bias the stack of markers toward the dispensing mouth of aperture 122. Hence, irrespective of the angle at which the dispenser is used, a marker will always be available at the dispensing aperture 122 and available for attachment. This can be particularly preferred when friction is used to transfer rotation between the dispenser and marker, e.g. marker 170, but may also be of benefit when marker 190 is used and which may be freely movable along the longitudinal axis of the dispenser.

It will be appreciated that the marker attachment feature is not limited to a threaded attachment formation. In other embodiments, an adhesive material may be provided on an underside of the marker. This also obviates the need for a threaded formation on the reference array arms. Instead, the marker may simply be stuck to the attachment site by the adhesive material on the marker. In this embodiment, a spacer may be provided between adjacent markers within the cavity 118 to prevent adhesive material of an upper marker contacting the detection part of a lower marker.

In other embodiments, a snap fit or push fit attachment feature may be provided on the marker. A corresponding push fit or snap fit feature may then be provided on the reference array and to which the marker may then be connected.

As illustrated in FIG. 12, instead of using an internal thread, an external thread 402 may be provided on the holder part 404 of the marker 408. In that case, the attachment sites on the reference array are in the form of a recess with an internal thread into which the markers 408 may be screwed.

Further, if no internal thread and recess is provided, then a spacer, for example an annular ring or similar, may be provided between each adjacent pair of markers 408 in order to separate adjacent markers and prevent an upper marker from damaging the detection part of a lower marker.

When using screw threads for the marker attachment feature, preferably the markers include an anti-rotation feature. As illustrated in FIGS. 7 and 8 the marker may include ribs which co-operates with grooves within the dispenser, but other arrangements are also possible. For example, one or more grooves may be provided in an outer surface of the holder, and which may cooperate with one or more corresponding anti-rotation features, e.g. one or more ribs, extending along the inner surface 115 of the inner wall 114 and in the direction of the longitudinal axis of the dispenser. As discussed above, these anti-rotation features cooperate to prevent rotation of the marker 170 relative to the dispenser when the dispenser is rotated.

In some aspect, multiple dispensers 100, each within a sterile packet, may be provided in a single container, such as a box or carton, and then dispensers taken from the container when required before or during a surgical procedure.

In other approaches, one or more dispensers may be provided as part of a kit of parts including one or more reference arrays and optionally one or more instruments to which the reference array may be attached in use. Hence, the dispenser may serve to both protect and package the reference markers and also provide a mechanism by which the markers may be more easily and reliably attached to the reference array when required.

In this specification, example embodiments have been presented in terms of a selected set of details. However, a person of ordinary skill in the art would understand that many other example embodiments may be practiced which include a different selected set of these details. It is intended that the following claims cover all possible example embodiments.

Any instructions and/or flowchart steps can be executed in any order, unless a specific order is explicitly stated. Also, those skilled in the art will recognize that while one example set of instructions/method has been discussed, the material in this specification can be combined in a variety of ways to yield other examples as well, and are to be understood within a context provided by this detailed description.

While the disclosure is amenable to various modifications and alternative forms, specifics thereof have been shown by way of example in the drawings and described in detail. It should be understood, however, that other embodiments, beyond the particular embodiments described, are possible as well. All modifications, equivalents, and alternative embodiments falling within the scope of the appended claims are covered as well.

1. A dispenser for dispensing and attaching individual markers to a reference array of a tracking system, the dispenser comprising:
a housing defining a cavity therein extending along a longitudinal axis of the housing and the housing having a dispensing end defining a dispensing aperture of the cavity;
a plurality of markers stacked within the cavity, each marker having a marker attachment feature by which the marker is attachable to an attachment site of a reference array, and wherein the dispensing end provides a retainer exerting a retaining force on a bottom most marker sufficient to retain markers within the cavity but permitting the bottom most marker to be released from the cavity via the dispensing aperture when the bottom most marker has been attached by its attachment feature to the attachment site of the reference array.

2. The dispenser of claim 1, wherein the retainer includes at least one sprung member.

3. The dispenser of claim 1, wherein the dispensing end is deformable to provide the retainer.

4. The dispenser of claim 1, wherein the marker attachment feature is one of a thread, an adhesive material, a push fit connection, or a snap fit connection.

5. The dispenser as claimed in claim 1, wherein the marker attachment feature is a thread, and each marker includes an anti-rotation feature which interacts with a feature of an inner wall defining the cavity to prevent rotation of the marker relative to the housing.

6. The dispenser as claimed in claim 5, wherein the anti-rotation feature comprises one or more male formations and the feature of the inner wall comprises one or more female formations extending along the longitudinal axis of the housing.

7. The dispenser as claimed in claim 6, wherein the or each female formation extends all the way to the dispensing aperture.

8. The dispenser as claimed in claim 5, wherein the dispenser includes a torque limiting mechanism permitting the dispenser to rotate relative to a bottom most marker when a torque being applied to the bottom most marker by the housing exceeds a limit.

9. The dispenser as claimed of claim 4, wherein the housing comprises an upper part and a lower part and wherein the upper part defines the cavity and wherein the lower part is movable along the longitudinal axis toward the upper part from a first position to a second position and wherein the lower part extends beyond the dispensing aperture in the first position and the lower part does not extend beyond the dispensing aperture in the second position.

10. (canceled)

11. (canceled)

12. The dispenser as claimed in claim 1, wherein a spacer is provided between each adjacent pair of markers.

13. The dispenser as claimed in claim 1, wherein each marker comprises a holder which provides the marker attachment feature and a detection part which is detectable by the tracking system.

14. The dispenser as claimed in claim 1, wherein each marker includes a circular, annular, spherical or hemi-spherical or spherical cap shaped reflector of infra-red radiation.

15. The dispenser as claimed in claim 1, further comprising a resilient element arranged to bias the plurality of markers toward the dispensing aperture.

16. The dispenser as claimed in claim 1, wherein an upper end of the housing includes a drive connection by which a drive tool can be attached to the dispenser.

17. (canceled)

18. (canceled)

19. The dispenser as claimed in claim 1, wherein the dispenser and the markers are sterile.

20. (canceled)

21. (canceled)

22. A method of attaching markers to a reference array for a tracking system using a dispenser holding a plurality of markers, the method comprising:
presenting a dispensing end of a dispenser holding a plurality of markers to an attachment site of the reference array;
attaching a bottom most marker within the dispenser to the attachment site using the dispenser, and
withdrawing the dispenser to release the distal most marker from the dispenser while retaining the marker attached to the attachment site.

23. The method of claim 22, further comprising repeating the method for each of a plurality of attachment sites of the reference array.

24. The method of claim 22, further comprising repeating the method for each of a plurality of reference arrays.

25. The method of claim 22, further comprising rotating the dispenser to attach the distal most marker to the attachment site.

26. (canceled)

27. The method of claim 22, and further comprising removing the dispenser from a sterile packet.

28. (canceled)
